(19) United States
(12) Patent Application Publication
(10) Pub. No.: US 2006/0122481 A1
(43) Pub. Date: Jun. 8, 2006

(54) System and method for location based remote services

(76) Inventors: Crispian Lee Sievenpiper, Waukesha, WI (US); Sandra Fieldstad Wiley, Eagle, WI (US)

Correspondence Address: Patrick S. Yoder;FLETCHER YODER, P.O. Box 692289, Houston, TX, 77269-2289, US

(21) Appl. No.: 10/994,978

(22) Filed: Nov. 22, 2004

(51) Int. Cl. A61B 5/05

(52) U.S. Cl. 600/407

(57) ABSTRACT

A system and method for locating and servicing a medical device. A portable computer system transmits a wireless signal to physically locate and identify the medical device which is disposed in proximity to the portable computing system. Further, the medical device may be wirelessly coupled to the portable computer system. The device may be serviced by a field engineer and/or a remote servicing center via the portable computing system and the wireless connection. An electronic toolset may be employed.

### BACKGROUND

The invention relates generally to the field of equipment services. More particularly, the invention relates to techniques for locating, identifying, and servicing medical systems within a medical facility or institution.

There are many different electronic devices available for learning about and treating patient conditions in the medical field. Over recent decades, more sophisticated systems have been developed that include various types of electrical data acquisition which detect and record the operation of systems of the body and, to some extent, the response of such systems to situations and stimuli. Even more sophisticated systems have been developed that provide images of the body, including internal features which could only be viewed and analyzed through surgical intervention before their development, and which permit viewing and analysis of other features and functions which could not have been seen in any other manner. All of these techniques have added to the vast array of resources available to physicians, and have greatly improved the quality of medical care.

However, many medical systems, such as medical imaging systems, are complex machines. As a result, they may require periodic servicing from a technician or service provider. Typically, a technician or field engineer (FE) may travel to a remote site such as a medical facility to service medical systems housed at the facility. However, specific medical systems may be difficult to locate and find in the medical facility. This may be especially problematic for stand-alone medical systems (and components) not connected to a network. For example, lack of connection of the medical system to a medical facility information system, a local area network, the Internet, and so on, may prohibit or make difficult identifying and servicing the medical system, on-site or remotely. In general, medical facilities and institutions, such as hospitals and medical complexes, may include several sub facilities and buildings, with thousands of medical components, devices, and systems housed in various rooms and buildings.

With movement of the medical system components, such as during shipping, warehousing, and installation of new or relocated components, as well as with mobile systems that may be stored in various rooms and locations, the field engineer may find it challenging to locate the medical system. Further, once the system or device is found, it may be difficult to identify relevant aspects of the device, such as service contractual information, current repair status, machine state and service history. Furthermore, as indicated, stand-alone devices not connected to a network and/or not configured to couple to an external processor-based system may not benefit from on-site/remote electronic services.

A need exists for a technique to reduce the time required to locate and identify medical systems and components within a medical facility. Further, a need exists for a technique to facilitate connection of medical systems and components to a network and/or server to promote efficient service and repair of the medical system. The technique should provide for both on-site and remote servicing of the medical systems and components.

### BRIEF DESCRIPTION

A system and method for locating and servicing a medical system or device within a medical facility is provided to respond to such needs. The technique can be implemented via a location-based system, such as an ad hoc network which may operate without access points and independent of resources remote from the medical facility. On the other hand, the technique may also utilize remote resources, such as the Internet, a remote service center, an on-line center, and so on.

To accommodate detection of a medical system component and the formation of a location-based network, the technique equips medical systems and components, such as imaging systems and their scanners and/or computers, with a passive or quasi-passive wireless communication device which responds in the presence of a transmitter carried by a field engineer. The passive wireless devices may operate on radio frequency (RF), optical, acoustic, and so on. When in close proximity, the medical system or component may be added to a field-engineer (FE) network and/or location-based (e.g., ad hoc) network. The field engineer can then perform service with a standard electronic toolset, for example. It should be noted that the passive or quasi-passive device may be more operational or active than passive RF tags containing identification information, for example. On the other hand, the device may be passive in the sense of not actively emitting a wireless signal unless responding to an authenticated wireless signal initiated by the FE transmitter. Such infrequent wireless emission and intermittent wireless connection enhances the efficiency and security of servicing and communicating with the medical system.

Aspects of the present technique provide for locating and servicing a medical device, including operating a portable computer system to transmit a wireless signal to detect the medical device, and wirelessly coupling the medical device to the portable computer system. The technique provides for locating, identifying, and servicing the medical device. Other aspects of a technique for locating and servicing a medical device include transmitting a wireless signal to locate the medical device, detecting the medical device via the wireless signal, and wirelessly coupling the medical device to a location-based network.

In one example, a medical system is maintained by transmitting a wireless signal from a portable computing system having a transmitter adapted to transmit the wireless signal, receiving the wireless signal via a passive or quasi-passive communication device disposed in the medical system, wirelessly coupling the portable computing system and the medical system, and servicing the medical system.

In yet another example, a medical system is maintained by transmitting a wireless signal from a portable device, receiving the wireless signal via a communication device disposed on a component of the medical system, wirelessly coupling the portable device with the component, and determining the physical location of the component.

In one embodiment, a service system includes a portable computing system having a transmitter component adapted to transmit a wireless signal to locate the medical system; a wireless communication device disposed in the medical system and repsonsive to the wireless signal; and a first connector disposed on the portable computing system and a second connector disposed on the medical device, wherein the first and second connectors are configured to facilitate a wireless network connection between the portable computing system and the medical device. The portable computing system may be adapted to service the medical system via the wireless network connection.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical representation of a service system for a medical device in accordance with an exemplary embodiment of the present invention;

FIG. 2 is a general diagrammatical representation of the service system of FIG. 1 depicting components of the service system in more detail in accordance with an exemplary embodiment of the present invention;

FIG. 3 is a diagrammatical representation of the service system of FIG. 1 in operation in accordance with an exemplary embodiment of the present invention;

FIG. 4 is a diagrammatical representation of a location-based service module disposed on a medical device and portable computer system in accordance with an exemplary embodiment of the present invention; and

FIG. 5 is a block flow diagram of a method for servicing a medical device utilizing a wireless connection and location-based service modules in accordance with an exemplary embodiment of the present invention.

### DETAILED DESCRIPTION

To accommodate detection of a medical system component and formation of a location-based network, the technique equips medical systems and components, such as imaging systems and their scanners, with a passive or quasi-passive communication device which responds in the presence of a transmitter. Again, such passive devices may operate on radio frequency (RF), optical, acoustic, and so on. A service provider or field engineer (FE) may carry the transmitter (e.g., laptop, transceiver, antennae, wireless network card, 802.11b device, etc.) to search for a response from the passive communication device (e.g., receiver, transceiver, antennae, wireless network card, 802.11b device, etc.) disposed on a component of the medical system or disposed in the medical system as an independent item. When in close proximity, the medical system or component may be added to a field-engineer network. The field engineer can then perform service with a standard electronic tool set, for example. Again, the passive device is more operational than strictly passive RF tags containing identification information, for example. On the other hand, the device does not actively emit a wireless signal, but instead responds to an authenticated wireless signal initiated by the transmitter. Such low activity of wireless emission and wireless connection of the medical device enhances efficiency and security.

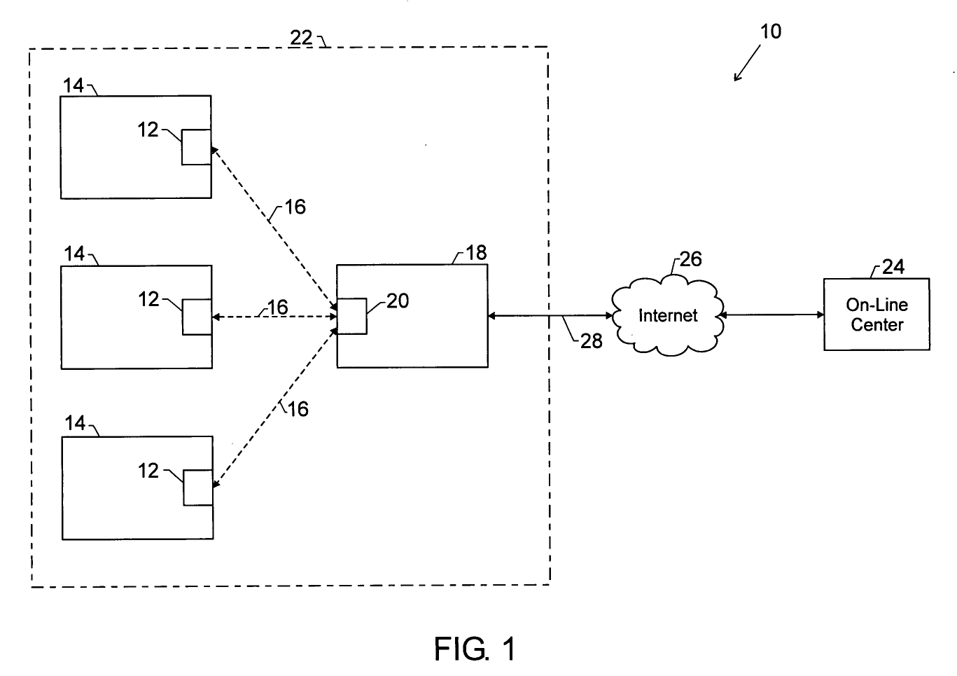

FIG. 1 depicts an exemplary service system 10 for medical devices such as imaging systems and their scanners. The service system 10 includes a quasi-passive or passive communication component or device 12 disposed on a medical device 14. The passive device 12 may be configured to provide for detection of the medical device 14, as well as for the wireless connection 16 of the medical device 14 to a portable computing system 18. A transceiver 20 disposed on the portable computing system 18 may transmit a wireless signal to detect the passive component 12 and to also provide for wireless connection 16 of the medical device 14 to the portable computing system 18. The transceiver 20 may represent multiple parts and include a transmitter antenna, a transceiver, a wireless IEEE 802.11b device or network card, a radio frequency (RF) device, an acoustical or optical device, and so on. The passive component 12 may represent multiple components and include a receiver, an antenna, a transceiver, a wireless IEEE 802.11b device or network card, and so forth.

The service system 10 may be used to locate and service a medical system or device 14 within a medical facility. The system 10 can be implemented as a location-based system 22, such as in an infrastructure mode and/or ad hoc mode. Infrastructure networks are traditional wireless networks requiring an access point. Ad hoc networks may operate without access points and independent of resources remote from the medical facility. In certain instances, a field technician may wish to access data or operating parameters from a scanner of the medical device 14, for example. Accordingly, a field unit or portable computing system 18, such as a laptop computer or hand-held device, may be linked to the controllers and electronic storage of the medical system or device 14 via the passive device 12. To improve portability of the network and to facilitate determining the physical location of the medical device 14, as well as to facilitate the efficient download/upload of information from the medical device 14, the field unit or portable computing system 18 may be configured to communicate with the device 14 controllers and storage via a wireless protocol, such as IEEE 802.11b, Bluetooth, RF communications, optical communications, acoustic communications, and so on. Advantageously, the field technician or field engineer, via the portable computing system 18, may be able to monitor operations of the medical device 14 (including a scanner, for example) and provide system adjustments in response to improve the quality of the data and images produced. Of course, the portable computing system 18 may also communicate with the medical system and devices 14 via other network connections.

The location-based system 22 (e.g., ad-hoc network) can be customized, and easily altered, for conformance with local, state and federal or other laws or regulations, particularity those relating to access to patient data. Moreover, the technique offers automatic or easily adapted compliance with hospital information system data access regulations, such that data can be flagged to insure privacy based upon the user or access method. Further, a user may be responsible for setting the security or access level for data generated or administrated by that user, or other participants may be responsible for such security and access control. Indeed, the portable computing system 18 can be programmed to implement default access levels for different types of users or user functions. In the illustrated embodiment, the passive component 12 provides for intermittent wireless connection to further advance network security. In other words, unlike active components or devices which actively emit a continuous or semi-continuous wireless signal, the passive device 12 may be configured to emit a signal only if initiated by the transceiver 20 on the portable computing device 18, for example.

The technique may also utilize remote resources of a remote service center 24 (e.g., on-line center) via the Internet 26 or other external network. The connection 28 to the Internet 26 from the portable computing system 20 may be wireless. In general, the portable computing system 18 may communicate with remote locations and devices via an external network, such as a Local Area Network (LAN), a Server Area Network (SAN), a Metropolitan Area Network (MAN), a Wide Area Network (WAN), a Virtual Private Network (VPN), the Internet 26, or any other suitable kind of network. Communications over the external network may be conducted via any number of communications schemes and protocols, such as Global Standard for Mobile (GSM), Time Division for Multiple Access (TDMA), Code Division Multiple Access (CDMA), Frequency Division Multiple Access (FDMA), radio frequencies (RF), or any other suitable communications techniques. Again, the external network may include remote monitoring or service centers 24, which may receive operation data, scanner data, imaging data from the portable system 18 and/or from the medical system 14 via the portable system 18. Advantageously, the remote service center 24, via the external network or Internet 26, may improve the performance of the medical system and scanner, such as improving the image data quality via monitoring and adjusting the operating parameters remotely. The external network or Internet 26 may also facilitate access to remote databases which may store large volumes of scanner data and operating data from a wide variety of sources coupled to the external network. That is, data from multiple medical scanners, for example, may be stored in a central location.

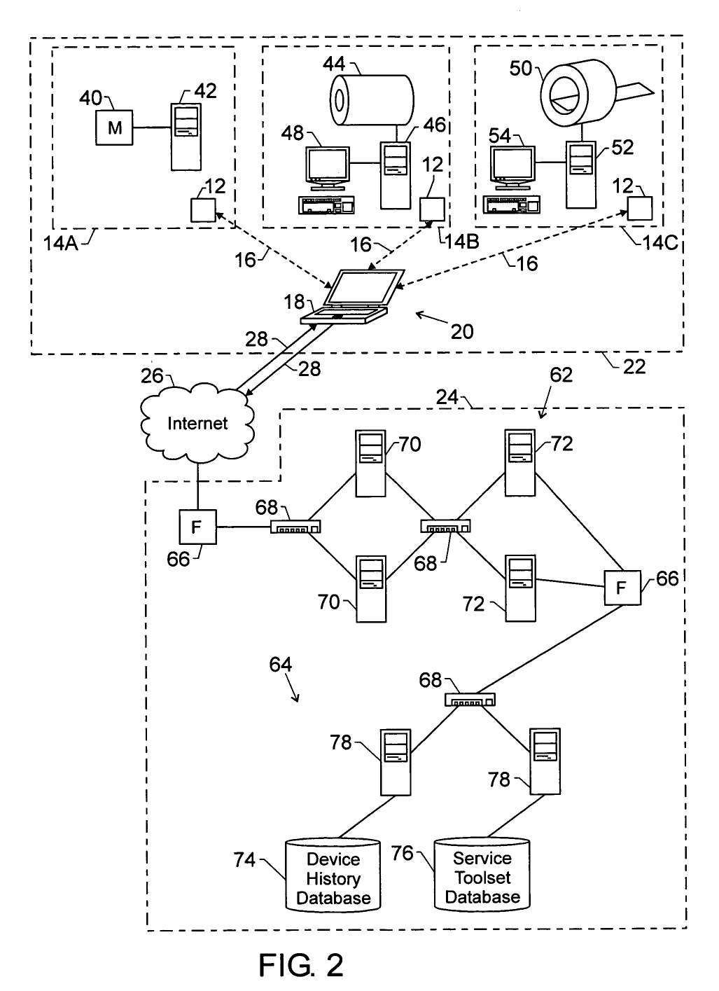

Referring generally to FIG. 2, the exemplary service system 10 is illustrated in more detail. Exemplary medical systems 14 that may benefit from the service system 10 include medical electronic systems 14A, such as medical imaging systems. In general, a medical electronic system 14A may include a sensing element 40 (e.g., scanner) and a server or workstation 42. A passive communication device 12 may be installed on the sensing element 40, the workstation 42, or other components of the medical system 14A to facilitate detection and service of the system 14A. Alternatively, the device 12 may be disposed adjacent a component of the medical system or device 14.

Periodically, a service may need to be performed on the medical system 14 by a service provider. For example, the services of a service provider may be retained to improve the images produced by a medical imaging system 14 (including systems 14A, 14B, and 14C). Furthermore, a service provider may be retained to repair or replace a defective component of the medical electronic systems 14A, 14B, and 14C. A service provider may also be used to upgrade old software or load new software into the medical electronic systems 14A, 14B, and 14C. In general, an electronic toolset disposed on the medical system 14, the portable computing system 18, and/or at a remote center 24, may be used to analyze and service the medical system 14.

As for the operation of the medical imaging system, the system 14 components generally include some type of imager (sensing element 40) which detects signals and converts the signals to useful data. In operation, imaging systems 14 are typically available for diagnosing medical events and conditions in both soft and hard tissue, for analyzing structures and function of specific anatomies, and in general, for screening internal body parts and tissue. In general, image data indicative of regions of interest in a patient are created by the imager either in a conventional support, such as photographic film, or in a digital medium. Ultimately, image data may be forwarded to some type of operator interface 42 or to a medical facility data network, for example, for viewing, storing, and analysis.

Typical imaging system components may include scanners 40, operator workstations and controls, viewing stations, servers 42, controllers, data processing stations, shielding devices, patient furniture and restraints, patient and technician protective equipment, and so on. Again, passive communication devices 12 may be disposed on one or more of the components of the medical imaging system to facilitate determining the location the components. It should be noted that the communication devices 12 may be installed at existing connectivity points of the medical imaging system (or generic medical system). In general, medical systems may include hardware and software that facilitates the electronic connection of the medical device to a service system.

The medical device 14 may have one or more connectivity points which could be utilized to connect the medical system 14 to a remote service center 24 where service may be performed remotely on the medical device 14. Typically, connectivity points may be disposed on the operator interface or computer of the medical system 14, as well as on the primary device of the system, such as scanner or scanner parts. Traditionally, the remote service center may service the medical device 14 with a standard electronic toolset, for example, if the connectivity point is utilized and the medical device is electronically connected to the remote service center 24. Such toolsets may be configured for specific systems or generalized for a given modality or a variety of modalities. In the case of a MRI device, a conductivity point may exist on the magnet and/or scanner, as well as on the MRI system computer itself, and so forth. Data may be stored in the MRI component at each conductivity point.

Servicing via the remote service center 24 may be problematic where the connectivity point of the medical device or system 14 is not connected to a network or the Internet. Indeed, for medical systems 14, such as mobile ultrasound systems, the medical system components are generally not connected to a network. These mobile systems may be placed in a room, for example, with several systems stored adjacent one another. Thus, such mobile systems and other stand-alone medical systems 14 may not benefit from service via a remote service center 24 or standard toolset, for example. Indeed, locating and identifying a single ultrasound sensor, for example, in a room full of stand-alone ultrasound systems may be difficult.

Thus, the present technique provides for a field engineer to travel to the medical institution or facility and to use a portable computing system 18 to detect and identify the various medical systems 14 including mobile systems and stand-alone systems. The portable computing system 18 may take advantage of existing connectivity points disposed on the medical devices 14. The portable system 18 may take advantage of additional communication components installed on the medical systems 14. The field engineer and the portable system 18 may establish a wireless network connection with the medical systems 14, and thus provide for an interface between the remote service center 24 and the medical system 14. Furthermore, the location-based network 22 may include the appropriate hardware and software to provide for standardization of communication protocols with the remote service center 24, as well as replicate and/or map the functionality of standard remote toolsets, for example. Accordingly, a mobile or stand-alone medical system 14 previously operating without the benefit of remote monitoring and service may now be monitored and served via the present technique. Further, the system 14 may benefit from local electronic service via the portable computer 18 without remote support.

An exemplary medical imaging system 14 includes a magnetic resonance imaging (MRI) system 14B having a scanner 44, a processor 46, and a workstation 48. The scanner 44 includes a primary magnet for generating a magnetic field. In operation, a patient is position against the scanner and the magnetic field influences gyromagnetic materials within the patient's body. As the gyromagnetic material, typically water and metabolites, attempts to align with the magnetic field, other magnets or coils produce additional magnetic fields at differing orientations to effectively select a slice of tissue through the patient for imaging. Data processing circuitry receives the detected MR signals and processes the signals to obtain data for reconstruction. The resulting processed image data is typically forwarded locally or via a network, to an operator interface for viewing, as well as to short or long-term storage.

Typical MRI system 14B components including the scanner 44, the processor 46, the workstation 48, and other parts, may comprise the passive communication device 12. Once the MRI system 14B component is located, the field engineer or technician may establish a wireless connection with the component and service the MRI system 14B via an electronic toolset dispose on the MRI system 14B, on the portable computing system 18, and/or at a remote center 24. The field engineer via the toolset, for example, may adjust operation of the magnets, such as calibrating the magnet shim of the MRI scanner 44, or adjust the operation of cryogen system cooling the superconducting magnet, such as altering operating set points of the magnet cryogen system, and so on. Existing points of connectivity in an MRI system 14B may include the MRI scanner, MRI magnet, MRI computer, and so forth.

Another example of a medical imaging system includes a computed tomography (“CT”) imaging system 14C having a scanner 50, a processor 52, and a workstation 54. For the example of CT, the basic components of a CT imaging system 14C and scanner 50 include a radiation source and detector. During an examination sequence, as the source and detector are rotated, a series of view frames are generated at angularly-displaced locations around a patient positioned within a gantry. A number of view frames (e.g. between 500 and 1000) may be collected for each rotation. For each view frame, data is collected from individual pixel locations of the detector to generate a large volume of discrete data. Data collected by the detector is digitized and forwarded to data acquisition and processing circuitries, which process the data and generate a data file accessible, for example on a medical facility data network.

Typical CT system components, such as the scanner 50 including the source and detector, the processor 52 or workstation, and other components may comprise a passive communication device 12. As with the MRI system 14B discussed above, the CT system 14C may be serviced by a field engineer or technician via the portable computing system 18 and wireless connection 16. Indeed, an electronic toolset configured for analyzing and repairing the CT operation may be disposed in the medical system 14C and/or the portable computing system 18. An existing point of connectivity may include, for example, the CT computer system. A field engineer via a wireless connection between the portable computing system 18 and the CT system 14C may evaluate the machine history, for example, of the CT system 14C. The field engineer (or a remote center 24) may evaluate, for example, the number of splices collected with the CT machine, or the shot noise of the CT exciter tube, and so on.

Yet other examples of imaging systems that may benefit from the present technique include x-ray imaging systems, positron emission tomography (“PET”) imaging systems, mammography systems, sonography systems, infrared imaging systems, nuclear imaging systems, and so forth. Again, a passive component 12 may be installed in or amongst the medical systems 14A, 14B, and 14C. In general, the present techniques are applicable to medical systems operable to produce an electronic image and other data of a test subject. Moreover, the techniques may be relatively more beneficial for medical systems having a large number of parts (inventory) and/or relatively mobile components.

Other modality acquisition systems 14A may benefit from a service system 10. Again, a passive communication device 12 may disposed on a component of the system 14A having a computer system 42 that may collect sensor 40 data. The configuration system 14A may include, for example, a variety of data collection systems designed to detect physiological parameters of patients based upon sensed signals. Resulting output data, such as waveforms or video, may be stored in the computer system 42 and/or at other repositories or storage sites linked to a medical facility data network for example. Again, the components of the system 14A may include a passive communication device 12 which facilitates the locating, warehousing, and servicing of the medical systems and components. It should be emphasized that once initiated, a passive device 12 or other communication device may become operational in the medical system and emit a wireless signal, as well as receive and transmit data from the medical device 14

Systems 14A may also represent electrical data resources and modalities, such as electroencephalography (EEG), electrocardiography (ECG or EKG), electrical impedance tomography (EIT), and so forth. Components typically include sensors or transducers, such as sensor/monitors 40, which may be placed on or about a patient to detect certain parameters of interest that may be indicative of medical events or conditions. Thus, the sensors 40 may detect electrical signals emanating from the body or portions of the body, pressure created by certain types of movement (e.g. pulse, respiration), or parameters such as movement, reactions to stimuli, and so forth. Again, a passive device 12 installed on such components may facilitate determining the location of the various components in a medical facility or institution.

Again, a portable computing system 18 is provided to enable a service provider to communicate with medical imaging systems 14 and, if desired, communicate with the service center 24. The computing system 18 may be a mobile desktop computer, a notebook computer, a personal digital assistant (“PDA”), or some other mobile communications device. In addition, the computing system 18 once wirelessly connected to the medical systems 14 may provide data automatically to the service center 24. The computing system 18 may be used to initiate and track service time. In addition, in the illustrated embodiment, the computing system 18 generate provide Global Positioning System (“GPS”) data. Such data may be fed to the on-line service system 24 to enable the service center 24 track and document the movements of the portable computing system 18 and the medical systems 14 (including systems 14A, 14B, and 14C).

As discussed, the service system 10 may also include a remote service center 24. The service center 24 enables a service provider to diagnose or repair problems associated with the medical system 14. The service center 24 may be coupled to the medical system or device 14 via a network (e.g., Internet 26) and the portable computing system 18. The configuration enables data to be transmitted from the medical systems 14A, 14B, and 14C to the service center 24. For example, medical images taken and stored by the medical imaging systems 14B and 14C may be transmitted via the portable computing system 18 and the Internet 26 to the service center 24 for analysis by a service provider. In the illustrated embodiment, the medical systems 14 may be self-aware, i.e., the medical systems 14 may store data and operate similar to a personal computer that has plug-and-play capability.

The service center 24 is a processor-based system that is operable to store and to process the service data from the medical electronic systems 14 and the field engineer via the portable computing system 18. In the illustrated embodiment, the service center 24 may begins collecting data for a specific service activity when the service center 24 receives a request for service. The collection of data related to the specific service activity may end when the field engineer indicates to the on-line center 24 that the service has been performed. Data or information may be compiled by the remote system 24 or by the field engineer on the portable computing system 18. Such data and information may describe the service performed and the service provider performing the service. For example, the service information may include the serial number of the medical imaging system and the service performed on the medical imaging system. The service provider data may include the time that it took the service provider to perform the service, billing information for the service provider, various expenses related to the service, and other pertinent pieces of data, such as the location of the service provider when the service was performed.

In the illustrated embodiment, the service center 24 comprises an application server system 62 and a database server system 64 that contains various types of medical system data for a plurality of different modalities. The application server system 62 is operable to process data from the medical systems 14, the portable computing system 18, and the database server system 64. The application server system 62 comprises firewalls 66 and hubs 68. In addition, the application server system 62 comprises a plurality of load balancers 60 and web servers 62. The load balancers 60 balance the data loads to and from the web servers 46. The web servers 62 store and execute the software applications that enable the application server system 62 to process the data.

The database server system 64 comprises a device history database 74 and a service toolset database 76. The device history database 64 is coupled by a server 68 to a hub 68. In addition, the service toolset database 76 is coupled by a server 70 to the hub 68. The hub 68, in turn, is coupled to the application server system 62. The device history data may be previous service data, previous part replacement data for the medical imaging system, the model number of the medical imaging system, and many other types of historic data related to the specific medical imaging system or class of medical imaging systems. In general, the service toolset database 76 may include code to analyze machine data, resolve problems, and identify potential solutions, associated with a plurality of different systems. In particular, the toolset database 76 may provide code, for example, that facilitates the update and revision of medical machine processor registers and configuration files, for example.

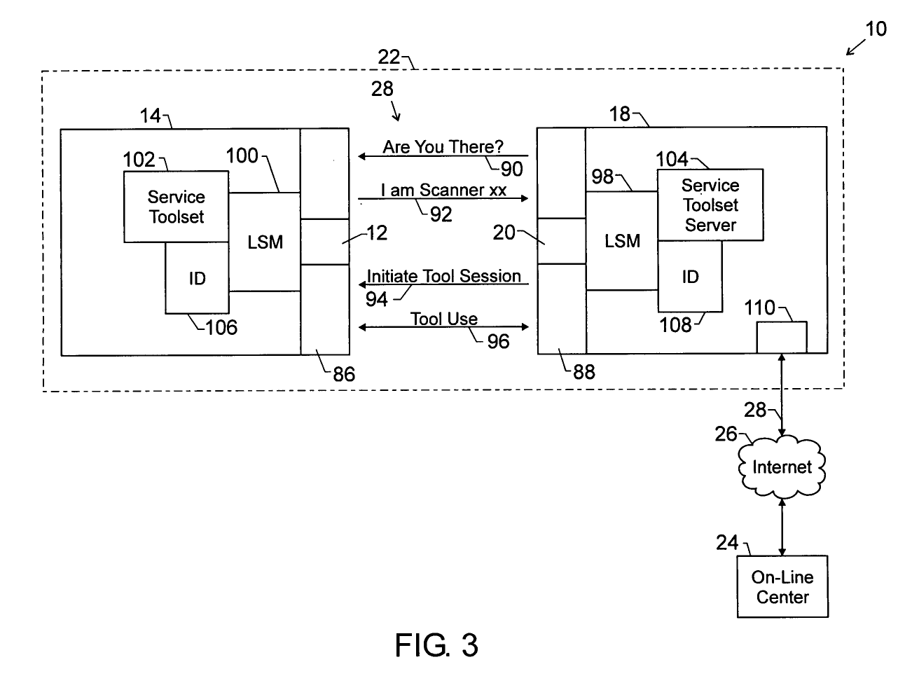

FIG. 3 depicts a location-based service network 22, and illustrates the interaction between a medical device or system 14 and the portable computing system 18. In one embodiment, the location-based network 22 comprises a wireless ad hoc network. As appreciated by those skilled in the art, ad hoc networks may demonstrate self-organization and scalability making for a robust solution. These networks can be built upon open protocols such as HTTP and SSL, and configured with wireless network 802.11b cards disposed on the medical device 14 and the portable system 18, for example. In operation, the exemplary portable system 18 may initially transmit a wireless signal in an effort to detect the medical device 14, as represented by reference numeral 90. Upon receipt of the wireless signal 28 by the communication device 12 disposed on the medical device 14, the quasi-passive component 86 may respond or alternatively, be read by the portable system 18 (e.g., via transceiver 20) to detect the presence and location of the medical device 14. Upon locating and identifying the medical device 14, a tool session may be initiated, as depicted by reference numeral 94. The tool session may be implemented over the wireless connection established via the exemplary wireless network cards. The electronic tools may include code for revising processor registers and configuration files of the medical device 14, for example.

Service system components include location-based services modules 98 and 100 disposed on the portable computer 18 and the medical device 14, respectively. Service components also include one or more service tool sets 102 disposed on the medical device 14 and/or portable computer 18. It should be noted that the portable computer 18 may act as a service tool set server 104 which may permit remote service from the on-line center 24 of the medical device 14. The portable computer 20 may also perform as a service tool set server 104 or multiple medical devices 14 wirelessly connected to the portable computer 18. Furthermore, service components may include identification information 106 and 108 stored on the respective devices. Generally, the medical device includes a quasi-passive component 86 including a communication device 12. Further, the portable device 18 generally includes a transceiver configuration 88 having a transmitter or transceiver 20.

In general, the LSM modules 98 and 100 provide for protocol definition and authentication. The modules 98 and 100 may complement each other and provide the hardware and software on both the medical device 14 and the portable computer 18 for authenticating, establishing, and maintaining the wireless connection between the medical device 14 and the portable computer 18. The modules 98 and 100 may provide functions similar to a TCP/IP stack, a protocol stack, and a server protocol stack, and so on. Moreover, the transceiver portion 88 may scan the ID 106 (e.g., RFID chip). The ID 108 on the portable system 18 may include memory comprising identification information of the portable system 18. Such ID 108 information may be utilized in the authentication process, for example.

As discussed, the location-based system 22 may utilize a wireless network having infrastructure modes that use one or more access points. In this configuration, the access points provide an interface to distribution system as the Ethernet, which enables wireless users to utilize corporate servers for internet applications, for example. In contrast, an optional feature with the 802.11 standard is an ad hoc mode, which allows a radio network interface card (NIC), for example, to operate in what an independent based service set (IBSS) network configuration. With an IBSS, there are no access points, and the user devices communicate directly with each other in a peer-to-peer manner. Further standards under development include, for example, the 802.11s standard which is a mesh network standard employing ad hoc techniques.

An ad hoc mode may allow a user to spontaneously form a wireless local area network. Through ad hoc modes, data may be transferred from one device to another, without an access point and cables. In general, an ad hoc network is a technique for wireless devices to directly communicate with each other. Operating in ad hoc mode allows all wireless devices within range of each other to discover and communicate in peer-to-peer fashion without involving central access points (including those built into broadband wireless routers). To set up an ad hoc wireless network, each wireless adapter may be configured for ad hoc mode versus the alternative infrastructure mode. In addition, the wireless adapters on the ad hoc network should use the same SSID and the same channel number. An ad hoc network may feature a small group of devices all in proximity to each other.

The 802.11 wireless standard, in general, is simply a protocol for communications on a wireless local area network (LAN). Popularly known as Wi-Fi (wireless fidelity), this IEE standard calls for the use of unlicensed 2.4 gigahertz (GHz) band, allowing anyone to build their own network, and anyone to employ their own wireless interface to such a network. Generally, there are two basic flavors of Wi-Fi, including an 802.11a which allows for a bandwidth of 1 to 2 mega baud per sec (Mbps), and 802.11b which uses a different modulation scheme and can support speeds of 5.5 and 11 Mbps. Wi-Fi has become a ubiquitous global standard for wireless data communications, with applications in offices, homes, campuses and so on.

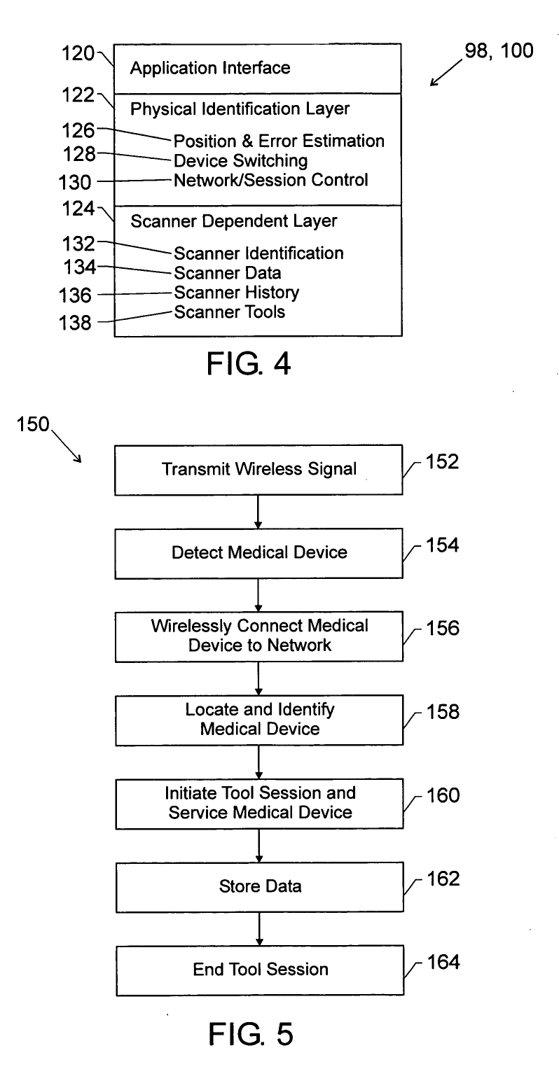

FIG. 4 is a diagrammatical representation of location-based service modules 98 and 100 disposed on a medical device 14 and portable computer 18, respectively. Again, the location-based service modules 96 and 100 are intended generally to complement each other. The LSM's 98 and 100 may include an application interface 120, which may provide for different protocols other than the standard internet connectivity. The application interface 20 may provide for communication of non-standard location-based network protocols with standard networking and internet protocols. For example, the application interface 120 may provide for use of a standard electronic toolset from a remote service center 24.

The modules 98 and 100 may include a physical identification layer 122 and a scanner dependent layer 124. In the physical identification layer 122, software code may be provided to facilitate the position and error estimation 126 of one or more medical devices 14. For example, in the case of several mobile medical devices 14 stored or positioned adjacent to each other, the physical identification layer may provide for identifying the respective medical devices 14 via GPS, triangulation, signal strength analysis, and so on. In general, the purpose of the physical identification layers is to identify the location and specific medical device 14 and its specific location space. The physical identification layer may include the appropriate software and hardware as needed. Further, device switching 128 may be implemented functionally, for example, such that more than one medical device may be communicated with simultaneously. Similarly, a network/session control 130 may provide for communication with multiple medical devices 14, as well as for initiation and control of the communication sessions. Moreover, more than one field engineer may work on a single medical device 14 via the appropriate device switching 128 and network session control 130.

Moreover, the scanner dependent layer 124 may be used to facilitate the identification and service of the respective medical device 14. The scanner dependent layer 124 may provide for scanner identification 132, scanner data 134, scanner history 136, and scanner tools 138. The layer 24 may store the data and/or provide an interface for the data. It should be emphasized that a scanner dependent layer 124 may be generalized as a medical device dependent layer to include components other than a scanner.

FIG. 5 is a block flow diagram of a method for servicing a medical device 14 utilizing a wireless connection 28 and location-based service modules 98 and 100. A wireless signal is transmitted from a portable computer 18 (block 152). The portable computer 18 may detect a medical device 14 having a communication device 12 (see FIGS. 1 and 2) responsive to the wireless signal, as depicted by block 154. Further, the portable computer 18 may make a wireless connection to the medical device or system 14 (block 156). Such a connection may be made, for example, with a transceiver/receiver, standard 802.11b wireless network cards, and so on. It should be noted that the medical device 14 may be located and identified before or after wireless connection is established (block 158). Upon wireless connection of the portable computing system 18 to the medical device 14, a tool session may be initiated and the medical device 14 serviced, as depicted in block 160. Furthermore, any relevant data collected before, during, or after the wireless connection may be stored (block 162). Such data may be stored, for example, in the medical system 14, on the portable computer 18, at a remote center 24, and so forth. Finally, the tool session may be ended by the field engineer, as depicted in block 164.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for locating and servicing a medical device, comprising:
operating a portable computer system to transmit a wireless signal to detect the medical device, wherein the medical device comprises a communication component responsive to the wireless signal;
wirelessly coupling the medical device to the portable computer system;
locating and identifying the medical device; and
servicing the medical device.

2. The method as recited in claim 1, wherein sevicing comprises servicing the medical device with an electronic toolset.

3. The method as recited in claim 1, wherein servicing comprises servicing the medical device from a remote service center via the portable computer system and via the wireless coupling of the portable computer system to the medical device.

4. The method as recited in claim 1, wherein the medical device comprises a medical imaging system.

5. The method as recited in claim 1, wherein the medical device comprises a mobile ultrasound system.

6. A method for locating and servicing a medical device, comprising:
transmitting a wireless signal from a transceiver disposed on a portable device to locate the medical device;
detecting the medical device via the wireless signal; and
wirelessly coupling the medical device to the portable device, wherein the medical device and portable device form a wireless network.

7. The method as recited in claim 6, wherein wirelessly coupling comprises forming an ad hoc wireless network between the medical device and portable device.

8. The method as recited in claim 6, comprising communicating with a remote service center via the portable device.

9. The method as recited in claim 6, wherein detecting comprises receiving the wireless signal at a communication component disposed on the medical device.

10. The method as recited in claim 6, wherein the portable device comprises a portable network server or a portable computing system, or a combination thereof.

11. A method of maintaining a medical system, comprising:
transmitting a wireless signal from a portable computing system having a transmitter adapted to transmit the wireless signal;
receiving the wireless signal via a wireless communication device disposed in the medical system;
wirelessly coupling the portable computing system and the medical system to form a location-based wireless network; and
servicing the medical system.

12. The method as recited in claim 11, comprising locating the device via the wireless signal.

13. The method as recited in claim 12, wherein servicing comprises replacing a part of the medical system.

14. The method as recited in claim 11, wherein wirelessly coupling comprises wiressly coupling the portable computing system to the medical device via wireless network cards disposed on the portable computer system and medical device, and wherein servicing comprises servicing the medical system via the wireless network.

15. The method as recited in claim 11, wherein servicing comprises using an electronic toolset via the wireless network.

16. A method of maintaining a medical system, comprising:
transmitting a wireless signal from a portable device;
receiving the wireless signal via a communication device disposed on a component of the medical system;
wirelessly connecting the portable device with the component; and
determining the physical location of the component.

17. The method as recited in claim 16, comprising establishing an ad hoc network between the portable device and the component.

18. The method as recited in claim 16, comprising servicing the component.

19. The method as recited in claim 16, comprising electronically coupling the component with a remote service center via the portable device.

20. The method as recited in claim 16, comprising communicating with the component from a remote site via the portable device.

21. The method as recited in claim 16, comprising communicating with a remote service center from the portable device.

22. The method as recited in claim 16, comprising servicing the component from a remote service center via the portable device and via the wireless connection between the portable device and the component.

23. A system for servicing a medical system, comprising:
a portable computing system having a transmitter component adapted to transmit a wireless signal to locate the medical system;
a wireless communication device disposed in the medical system and responsive to the wireless signal;
a first connector disposed on the portable computing system and a second connector disposed on the medical device, wherein the first and second connectors are configured to facilitate a wireless network connection between the portable computing system and the medical device,
wherein the portable computing system is adapted to service the medical system via the wireless network connection.

24. The system as recited in claim 23, wherein the communication device comprises a wireless antenna receiver.

25. The service system as recited in claim 23, wherein the transmitter component comprises the first connector.

26. The service system as recited in claim 23, wherein the wireless communication device comprises the second connector.

27. The system as recited in claim 23, wherein the first and second connectors comprise wireless network cards.

28. The service system as recited in claim 23, wherein the medical system comprises a medical imaging system.

29. The service system as recited in claim 23, wherein the medical system comprises a mobile medical system.

30. A service system for a medical device, comprising:
means for transmitting a wireless signal to locate the medical device;
means for locating and identifying the medical device via the wireless signal;
means for wirelessly connecting the medical device to the means for transmitting the wireless signal; and
means for servicing the medical device via the wireless connection.

31. A computer program, comprising:
programming instructions stored in a tangible medium, wherein the programming instructions enable transmission of a wireless signal from a portable computing system to a receiver disposed on a medical device, and enable the wirelessly coupling of the medical device to the portable computing system, wherein the programming instructions facilitate locating the medical device.

32. The program as recited in claim 31, wherein the programming instructions enable the portable computing system to perform as a server in an ad hoc wireless network coupling the medical device to the portable computing system.

33. The program as recited in claim 31, wherein the programming instructions enable the portable computing system to perform as a server in a location-based wireless network coupling the medical device to the portable computing system.

34. The program as recited in claim 31, wherein the programming instructions enable the portable computer system to service the medical device via the wireless connection.
